(19) United States
(12) Patent Application Publication
(10) Pub. No.: US 2024/0320598 A1
(43) Pub. Date: Sep. 26, 2024

(54) USER PERFORMANCE EVALUATION AND TRAINING

(71) Applicant: KONINKLIJKE PHILIPS N.V., EINDHOVEN (NL)

(72) Inventors: Seyedali Sadeghi, Melrose, MA (US); Shyam Bharat, Arlington, MA (US); Claudia Errico, Medford, MA (US); Jochen Kruecker, Andover, MA (US); Hua Xie, Cambridge, MA (US)

(21) Appl. No.: 18/574,164

(22) PCT Filed: Jun. 20, 2022
(86) PCT No.: PCT/EP2022/066664

§ 371 Date: Dec. 26, 2023

Related U.S. Application Data
(60) Provisional application No. 63/215,642, filed on Jun. 28, 2021.

(51) Int. Cl. G06Q 10/0639

(52) U.S. Cl. CPC G06Q 10/06393

(57) ABSTRACT

A graphical ultrasound user evaluation tool is described. The evaluation tool employs a predictive model and log files recorded by the ultrasound scanner to determine one or more ultrasound user performance scores. The log files may be processed to extract actual (or recorded) performance metrics from the information (e.g., timed events such as button clicks) recorded in the log file, which are compared against predicted (or expected) metrics to determine the one or more performance scores. The predicted metrics may be obtained from a predictive model, which may be implemented by an analytical (e.g., regression or other) model or by a trained neural network. The ultrasound user performance scores are then graphically presented in a user-friendly manner, e.g., on a graphical dashboard which can provide a summary screen and further detailed reports or screens, responsive to user input, and/or update the scores based on comparison with a user-specified ultrasound user experience level.

### TECHNICAL FIELD

The present disclosure relates generally to medical imaging, such as ultrasound imaging and more specifically, to a quantitative graphical evaluation tool for evaluating a ultrasound user's performance.

### BACKGROUND

Ultrasound imaging has become ubiquitous for medical diagnostics, treatment monitoring, assistance for minimally-invasive procedures and in other clinical contexts/needs. Ultrasound imaging is highly dependent on operator skill and objective or uniform means for evaluating a ultrasound user's performance (e.g., workflow efficiency) are not generally available. Existing ultrasound systems, while capable of informing the user of the overall duration of an exam (from start to finish), are not equipped to provide any “quality of exam” metrics of the ultrasound user's performance. In most hospital settings, there is no well-accepted and intelligent tool/method for the ultrasound user's performance review and efficiency assessment. Having an accurate performance assessment tool is important for lab managers since it allows them to have accurate monitoring of staff performance and plan and balance staff assignments more efficiently.

### SUMMARY

A ultrasound user performance evaluation system according to some embodiment of the present disclosure includes a display, and a processor in communication with the display and at least one memory comprising computer-readable instructions which when executed cause the processor to generate one or more ultrasound user performance scores associated with a ultrasound user, the one or more ultrasound user performance scores being based, at least in part, on information recorded in an ultrasound machine log file resulting from an ultrasound exam performed by the ultrasound user with an ultrasound scanner, and provide a ultrasound user performance dashboard configured to graphically represent the one or more ultrasound user performance scores. In some embodiments, the processor, display, and the memory are part of a workstation of a medical institution, which is communicatively coupled, via a network, to a plurality of ultrasound scanners of the medical institution to receive respective ultrasound machine log files from any one of the plurality of ultrasound scanners. In some embodiments, the processor, display, and the memory are integrated into an ultrasound scanner. In some embodiments, each ultrasound user performance scores includes a numerical score and the ultrasound user performance dashboard is configured to the display numerical score, or a graphic representing the numerical score together with or instead of the numerical score. In some embodiments, the ultrasound user performance dashboard includes a graphical user interface (GUI) screen divided into at least a first display area for displaying ultrasound user performance scores associated with exam efficiency and a second display area for displaying ultrasound user performance scores associated with anatomical information efficiency. In some embodiments, the GUI screen includes a third display area that display customized ultrasound user feedback, the feedback customized based on the one or more ultrasound user performance scores.

In some embodiments, the processor provides the ultrasound machine log file as input to a trained neural network and obtains the one or more ultrasound user performance scores as output from the trained neural network. In other embodiments, the processor is configured to pre-process the ultrasound machine log file to determine actual ultrasound user performance metrics associated with the ultrasound user from the ultrasound machine log file. In such embodiments, the processor further obtains predicted ultrasound user performance metrics from a predictive model, which may be implemented in some embodiments by a trained neural network, compares the actual performance ultrasound user metrics with the predicted ultrasound user performance metrics to generate the one or more ultrasound user performance scores. In some embodiments, the neural network may be trained to generate the predicted performance metrics based on one or more one or more clinical context parameters, which may be selected from patient age, patient body mass index (BM), patient type, nature or purpose of the ultrasound exam, and model of the ultrasound scanner. In some embodiments, the neural network may additionally or alternatively receive the log file and determine clinical context parameters based on the information in the log file. In some embodiments, the predictive model (e.g., a neural network) may be configured to generate a respective set of predicted ultrasound user performance metrics for each of a plurality of different ultrasound user experience levels, which may be specified by the user (e.g., via the ultrasound user performance dashboard). In some embodiments, the performance metrics may include any combination of total idle time, total dead time, total exam time, total patient preparation time, total number of button clicks, total number of button clicks of a given button type, and total number of acquisition settings changes. In some embodiments, the ultrasound user performance dashboard provides one or more user controls for controlling the information presented via the dashboard, for example, the number and types of scores or detailed metrics, the ultrasound user and/or evaluation period for which scores are determined/presented, etc. In some embodiments, the dashboard is configured to display, upon user request, the actual and the predicted metrics concurrently (e.g., side by side). In some embodiments, the dashboard is configured to update the predicted metrics and/or the ultrasound user's performance scores(s) responsive to a user selection of a ultrasound user of a different experience level.

A method of providing performance evaluation of a ultrasound user according to some embodiments herein may include receiving, by a processor in communication with a display, an ultrasound machine log file. The log file and/or clinical context parameters are provided to a predictive model, and using output from the predictive model, the processor determines one or more ultrasound user performance scores. The ultrasound user performance scores are thus based at least in part on the information recorded in the log file. In some embodiments, the method involves providing the clinical context parameters to a trained neural network to obtaining predicted performance metrics, determining, by the processor, actual performance metrics of the ultrasound user from information recorded in the ultrasound machine log file, and comparing the actual performance metrics to corresponding ones of the predicted performance metrics to generate the one or more ultrasound user performance scores. The method further includes a graphical representation of the one or more ultrasound user performance scores on a display, such as in one or more user interface (GUI) screens as previously described. The GUI screens are part of a ultrasound user dashboard which is configured, in some embodiments, with one or more user controls or widgets for controlling the information presented on the dashboard and/or invoking additional function of the dashboard (e.g., events details and/or training screens).

Additional aspects, features, and advantages of the present disclosure will become apparent from the following detailed description.

### BRIEF DESCRIPTION OF THE DRAWINGS

Illustrative embodiments of the present disclosure will be described with reference to the accompanying drawings, of which:

FIGS. 1A and 1B show example ultrasound exam timelines, as recorded in log files, of a relatively less experienced user and a relatively more experienced user, respectively.

FIG. 2 shows an operational environment of a ultrasound user performance evaluation system according to the present disclosure.

FIG. 3 is a block diagram of an ultrasound imaging system which may embody a ultrasound user performance evaluation system according to the present disclosure.

FIG. 4 is a block diagram of components of a ultrasound user performance evaluation system according to the present disclosure.

FIG. 5 is an example of an ultrasound exam timeline and exam phases.

FIG. 6 is a graphical user interface of a ultrasound user performance dashboard according to embodiments of the present disclosure.

FIGS. 7 and 8 show additional graphical user interface screens associated with the ultrasound user performance dashboard according to the present disclosure.

FIG. 9 is a block diagram showing training and deployment stages of a neural network that can implement the predictive model of the ultrasound user performance evaluation system herein.

FIG. 10 shows a block diagram of components of a ultrasound user performance evaluation system according to further embodiments of the present disclosure.

FIG. 11 is a block diagram of an example processor in accordance with the principles of the present disclosure.

FIG. 12 is a flow diagram of an example process in accordance with the principles of the present disclosure.

### DETAILED DESCRIPTION

For the purposes of promoting an understanding of the principles of the present disclosure, reference will now be made to the embodiments illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. In particular, it is fully contemplated that features, components, and/or steps described with respect to one embodiment may be combined with features, components, and/or steps described with respect to other embodiments of the present disclosure. The numerous such combinations may not be described separately herein for the sake of conciseness and clarity.

Consistent imaging according to best practices for diagnostic ultrasound is important for maintaining a quality and efficient workflow. The disclosed systems and methods aim to address the lack of intelligent, well-accepted and objective tools for evaluating ultrasound user's performance, which can further provide opportunities and tools for training that would ultimately improve performance and lead to greater consistency in imaging in a given organization. Ultrasound user performance may be dependent upon upstream clinical context, such as the patient type, the reason of the exam, etc., and typically such context is not readily considered in conventional ways of evaluating ultrasound user's performance. There is, thus, a need for establishing a standardized quality assessment tool in the highly operator-dependent world of ultrasound imaging, which should ideally be user-friendly and provide the type of information and details to facilitate improvement of performance. The implementation of a performance review and/or educational tool within a diagnostic ultrasound department can benefit the ultrasound users as well as lab directors to improve the operational efficiency of the institution. Unbiased evaluations are critical to performance feedback and workflow optimization, all of which contribute significantly to the lab's financial performance and clinical quality. Clinical data analysis has shown that workflow-related factors in ultrasound imaging, such as exam duration, varies according to multiple clinical factors (e.g. the patient's length of hospital stay, BMI, age, diagnosis, the reason for exam, and model of ultrasound), which are independent of ultrasound user performance. Therefore, the clinical context may be a relevant factor to be take into account in the ultrasound user's performance assessment.

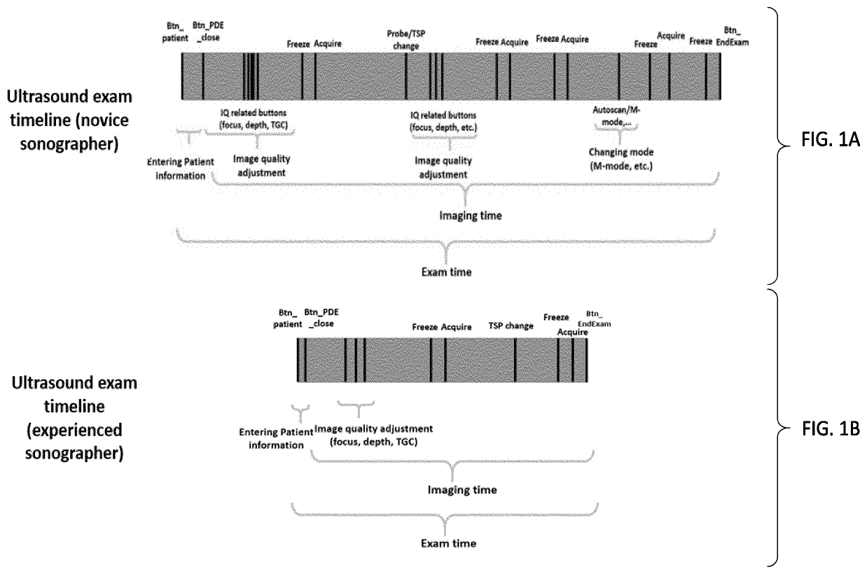

The service log files of an ultrasound imaging device (referred to here as ultrasound machine log files or simply log files) offer an enhanced set of attributes that are not usually available in the radiological information system (RIS) or the picture archiving and communication system (PACS) which are typically used to store patient image data or other diagnostic information. Service log files provide the entire narrative related to the users' workflow and imaging journey during ultrasound exams. Service log files can thus provide insight into whether a user was struggling to find the right imaging parameters, such as may be evidenced by changes in Probe/tissue-specific preset (TSP), exam length, choosing additional modes during an exam, changes in gain, etc. The extracted log information together with upstream clinical context may help unbiased assessment of ultrasound users' performance and may help identify challenges faced by the ultrasound user during image acquisition so the ultrasound user can improve their workflow efficiency in the image acquisition process. Illustrations of typical ultrasound exam timelines for a less experienced and more experienced user are shown in FIGS. 1A and 1B, respectively. As used herein, the term ultrasound user may refer to sonographer as is not limited by the certification or title of the ultrasound user unless otherwise indicated. Additionally, reference made to sonographers in the figures may also refer to any ultrasound user, regardless of certification status or geography unless otherwise indicated.

Referring to the timeline in FIG. 1A, the full complexity of workflow events in an exemplary exam for a novice ultrasound user can be appreciated. The timeline of any ultrasound imaging exam (or exam workflow) is captured by the various log file attributes recorded in the ultrasound machine log file. For example, the log file may record various events, each uniquely identified by an event ID and each uniquely associated with an event time (e.g., a timestamp that includes the time and optionally date when the event was logged). Thus, all button clicks or pushes, which is collectively used to refer to any user-machine interaction such to adjust the settings of the ultrasound machine and operate the ultrasound machine, are recorded and uniquely associated with a respective event time. The logged information can thus be mined to extract relevant and rich information relating to a particular exam, base on which ultrasound user performance can be evaluated, and preferably quantified. The different phases of the exam can be identified, and their duration determined, from the information recorded in a log file for use in evaluating the ultrasound user's performance. Further relevant information that can be extracted from the log files, in addition to the duration of each phase, can include number and frequency of changes of probe, TSPs or other settings (as can be captured by recording of button presses/selections), and number of image acquisitions, freezes, and the modes selected during imaging, etc. Information extracted from log files associated with different patients and clinical contexts can, thus, aid in identifying workflow issues and customization of the sequence of operations per protocol, which can be useful for evaluating exam efficiency estimation, such as by comparing a novice ultrasound user's workflow with the expected workflow of an experienced ultrasound user. Disclosed herein is an evaluation and training tool, which preferably includes a graphical component, that generates one or more performance scores of a ultrasound user's performance using the ultrasound user's service log files and upstream clinical context. Additionally, when implemented as a training tool, ultrasound users may receive information about the possible patterns of their scanning routines, within a chosen time frame, in the visualization review tool. This information helps junior ultrasound users optimize their workflow by comparing it with an expected workflow of ultrasound users with varying levels of experience.

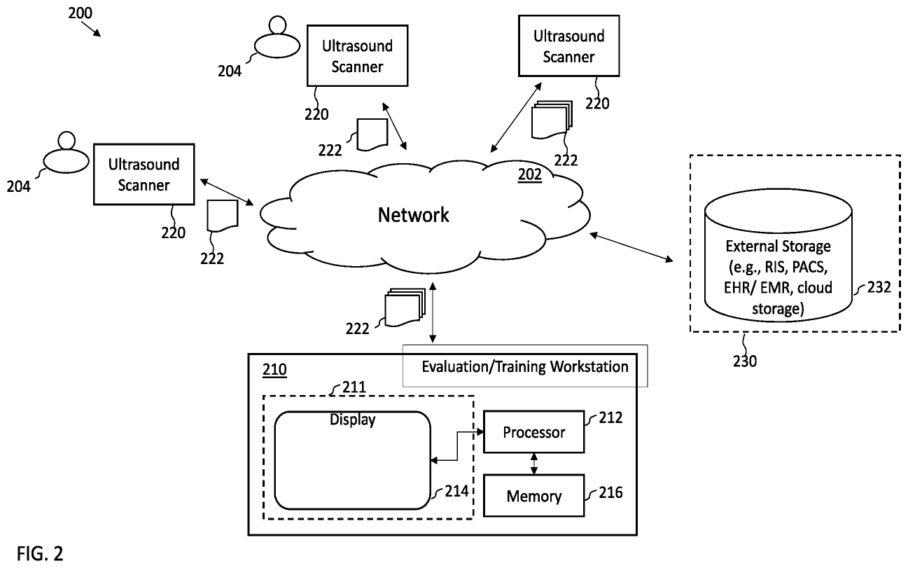

FIG. 2 shows an operational environment and system 200 according to the present disclosure. In the system 200, a computing workstation (also referred to as evaluation workstation) 210 is shown, which implements a ultrasound user performance evaluation tool according to the present disclosure. The workstation 210 is shown in the operational environment in FIG. 2 communicatively connected, via a network 202, to one or more ultrasound machines or scanners 220 and/or an external storage device 232. The one or more ultrasound scanners 220 are each configured to perform ultrasound imaging and to record, in local memory on the ultrasound scanner 220, respective log files 222 associated with each ultrasound exam performed by a user 204 using the ultrasound scanner 220. The operational environment of system 200 may represent a medical institution (e.g., a hospital, a clinical lab, an out-patient treatment facility, a medical training facility, a research lab or other research entity, or any other medical institution or organization that employs ultrasound imaging devices). As such, the ultrasound scanners 220 may be owned or otherwise affiliated with the medical institution. The medical institution may administer the ultrasound user performance evaluation tool on one or more evaluation workstations 210, also owned or otherwise affiliated with the medical institution. The workstation(s) 210 may be specifically configured to perform ultrasound user evaluation and/or provide training to a ultrasound user in accordance with performance criteria and evaluation standards associated with that medical institution.

The ultrasound scanner(s) 220, external storage device(s) 232, and the evaluation workstation 210 may be communicatively connected via any suitable wireless or wired network or any combinations thereof (e.g., a LAN and/or a WiFi network, or others). In some embodiments, the external storage device(s) 232 may contain patient medical records (e.g., EHR/EMR) and/or be part of the institution's Picture Archiving and Communication System (PACS). The one or more external storage device(s) 232 may be co-located, e.g., in a server room located at or affiliated with the medical institution, and may be connected via a gateway workstation 230, to the network 202. In some embodiments, one or more of the external storage device(s) 232 may reside in the cloud. The network 202 may operatively connect each of the networked devices (e.g., each of the ultrasound scanners 220, each evaluation workstation 210) to the storage devices 232 such that each networked device may transmit and retrieve data to the storage devices 232. For example, the ultrasound scanners 220 may transmit service log files 222 to the external storage devices 232 and the ultrasound scanner service log file(s) may subsequently be provided to the evaluation workstation 210 by the external storage devices 232 rather than directly from the scanner that generated it. Similarly, other data such as medical images may be stored in the external storage device(s) 232 and retrieved or accessed by the evaluation workstation 210 for implementing the ultrasound user performance evaluation tool. The evaluation workstation 210 includes a processor 212, a display 214, and memory 216, which may be implemented by any suitable number and/or combination of non-volatile memory devices. While referring to a one of a given hardware component (e.g., a processor, a display, a memory), it will be understood herein that the functions described with reference to that hardware component may be distributed among multiple such components (e.g., a plurality of processors, a plurality of memory devices, etc.) without departing from the context and scope of the present disclosure. The memory 216 stores computer-readable instructions, which when executed by the processor 212 cause the processor 212 to perform one or more processes associated with the graphical ultrasound user performance evaluation tool described herein.

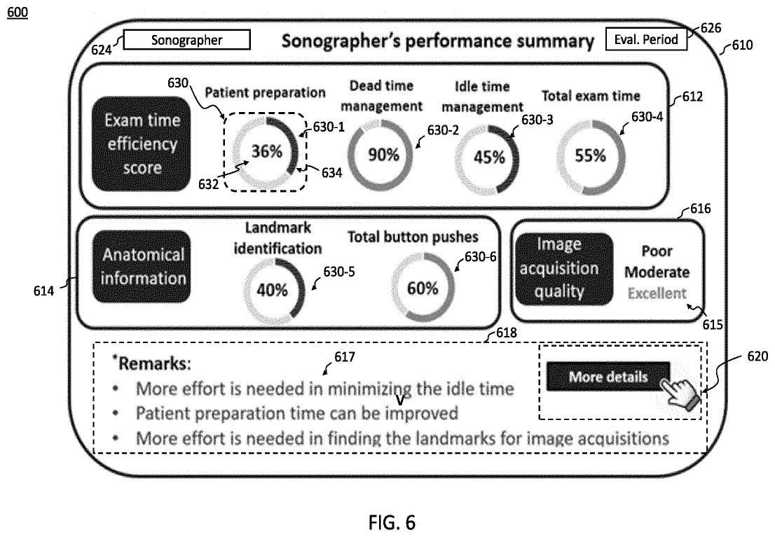

When executing the ultrasound user performance evaluation tool, the processor 212 generates one or more ultrasound user performance scores for a particular ultrasound user based, at least in part, on information recorded in an ultrasound machine log file generated responsive to an ultrasound exam performed by that ultrasound user. In addition, the processor 212 displays a ultrasound user performance dashboard, such as responsive to a user request, in which the one or more ultrasound user performance scores are graphically represented. In some embodiments, each of the ultrasound user performance scores comprises a numerical score and the ultrasound user performance dashboard may be configured to graphically represent the numerical score in addition to the numerical score, e.g., as shown in FIG. 6, or it may display the graphical representation instead of (or without) displaying the numerical score. In some embodiments, the processor 212 implements or communicates with a predictive model to generate the one or more ultrasound user performance scores. The processor 212 may provide the log file associated with the particular ultrasound user being evaluated to the predictive model and the predictive model may output the performance score(s) based on the information recorded in the log file. This may be achieved by a neural network trained, using multitude (e.g., hundreds or thousands) of recorded log files from expert ultrasound users in a given institution, to output any desired number or categories of performance scores(s) when presented with any new (not previously seen) log file.

In some embodiments, actual performance metrics of a particular ultrasound user, may be determined or extracted (e.g., by processor 212) from the information recorded in the log files (e.g., the recorded workflow constituting the collection of events or clicks and associated times). The actual performance metrics, which may also be referred to as the recorded metrics, may be compared (e.g., by the processor 212) to predicted performance metrics, which are metrics generated by a predictive model and correspond to the expected performance of a ultrasound user of a given experience level. In some embodiments, the system enables the user to select the ultrasound user level against which the actual (or recorded) metrics are compared for determining the performance score(s). As used herein “performance metrics” refers to any quantitative information (e.g., a numerical value) about user-machine interaction events recorded in the log file such as the total number of different types of button pushes or click, settings adjustments, probe selections or changes, and time or duration associated with each or elapsed between successive button pushes of certain types. As will be discussed further below, some examples of performance metrics may include, but are not limited to, total idle time, total dead time, total exam time, total patient preparation time, total number of button clicks during the exam, total number of button clicks of a given button type (e.g., total number of acquire or freeze events), and total number of acquisition settings changes.

The predicted performance metrics may be obtained from a predictive model, which may be implemented by any suitable analytical model (e.g., a regression analysis model) or by any suitable neural network trained to predict the desired set of performance metrics for a ultrasound user at a given (e.g. specified) experience level. The neural network may be trained to predict the performance metrics from different inputs. In some embodiments, the neural network may receive the current/new log file and/or upstream clinical context parameters associated with the exam workflow captured in the log file. In some embodiments, the neural network may be trained to predict the output based on an input log file alone. In yet other examples, the neural network may be trained to receive a set of clinical context parameters and to output the set of performance metrics that are expected from a ultrasound user of a specified experience level. As used herein, “clinical context parameters” or “upstream clinical context” may be used interchangeably and may include or be based on any of the type of ultrasound scanner used for the exam (also referred to as the model of the ultrasound scanner, example of which are Epiq 5 or Affiniti 70 ultrasound scanners manufactured by PHILIPS), the type of exam being performed (e.g., pulmonary, cardiac, abdominal, etc.), and various patient-specific information, non-limiting examples of which may include patient weight, height, body mass index (BMI), age, underlying health condition(s), clinical history, reason for exam, type of patient (i.e. inpatient/admitted or outpatient) and combinations thereof. Some or all of the information constituting the upstream clinical context may be retrieved from the log file(s) and/or from external systems (e.g., PACS, EHR/EMR, RIS, etc.) such as based on information included in the log file (e.g., patient name or ID).

While referring here to a neural network, it will be understood that in some embodiments a combination of neural networks may be used to predict the performance scores. For example, the neural network may be implemented a set of neural networks operatively arranged. For example, one or a plurality of neural networks may be trained to predict at least one performance score (e.g., one or more numerical scores) from a multi-variable input of clinical context parameters. In combination with the former, another neural network may be trained to predict, e.g., from one or more input images, another performance score, which may be a qualitative score, such as a classification of the input as “poor,” “good” or “excellent” or any other suitable set of categories. The latter may be used to score the ultrasound user's performance as to image quality. In yet other examples, one or more predictive functions of the predictive model (e.g., as relating to numerically scoring the ultrasound user's performance) may be performed by one or more analytical models while one or more other functions (e.g., image quality evaluation) may be performed by a neural network (e.g., a convolutional neural network) trained to operate on images as inputs. Various combinations and arrangements may be used for the predictive model of the present invention.

As will be further described, the ultrasound user performance evaluation tool may be embodied on the ultrasound machine itself, such as to enable the ultrasound user or a supervisor to launch the evaluation application and associated dashboard on the scanner itself, e.g., after the completion of an exam. The ultrasound user performance evaluation tool may be implemented on a standalone workstation that is separate from (e.g., remotely located, such as in a different room or wing of the medical institution, or in a different building from the scanner that generated the log file), and evaluation of the ultrasound user's performance may in such instances occur at some later time (e.g., another day, week, or month) after the completion of a particular exam. Various use case scenarios are envisioned that can advantageously employ the examples presented herein.

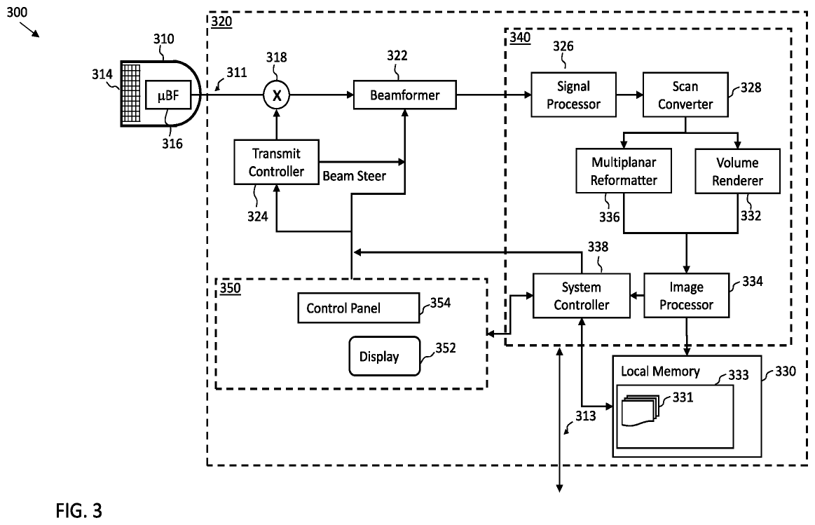

FIG. 3 shows a block diagram of an ultrasound imaging system (or scanner) 300, which can implement any of the ultrasound scanners 220 of FIG. 2. In some embodiments, the graphical ultrasound user performance evaluation tool according to the present invention may additionally or alternatively be implemented directly on the ultrasound scanner 300. In such embodiments, the processor, display and memory of workstation 210 are part of the ultrasound scanner and are configured, upon request, to process service log file(s) generated by that scanner for providing the graphical performance evaluation interface to the ultrasound user or another user, directly on the display of the scanner.

The ultrasound imaging system (or scanner) 300 includes electronic components which are configured to cause the transmission and reception of ultrasound signals and to perform signal and image processing for generating ultrasound images therefrom. At least some of the electronic components of the system 300 are be provided in a main processing portion 320 of the ultrasound scanner, also referred to as base or host 320 of the ultrasound scanner. During imaging, the base 320 is communicatively connected to an ultrasound transducer 310 via communication link 311, which may be implemented by a wired connection (e.g., serial, USB or other cable) or a wireless link. The system 300 includes a processor 340, which performs functions (e.g., signal and image processing of acquired data) associated with generating ultrasound images according to the present disclosure. As previously mentioned, and while referring herein to a processor, it will be understood that the functionality of processor 340 may be implemented by a single or a plurality of individual components (e.g., a plurality of individual processing units) operatively configured to perform the functions associated with processor 340. For example, processor 340 may be implemented by one or more general purpose processors and/or microprocessors configured to perform the tasks described herein, application specific circuits (ASICs), graphical processing units (GPUs), programmable gate arrays (FPGAs) or any suitable combinations thereof. Any of the processors of system 300 (e.g., processor 340) may implement the processor 212 of the evaluation workstation 210.

The system 300 also includes a user interface 350 which enables a user to control the ultrasound system 300. The user interface 350 includes a control panel 354, which may include any suitable combination of mechanical or hard controls (e.g., buttons, switches, dials, sliders, encoders, a trackball, etc.) and/or soft controls, such as a touch pad and various graphical user interface (GUI) elements that may include any suitable combination of menus, selectable icons, text-input fields, and various other controls or widgets, provided on a touch-sensitive display (or touch screen). The user interface 350 may include other well-known input and output devices. For example, the user interface 350 may optionally include audio feedback device(s) (e.g., alarms or buzzers), voice command receivers, which can receive and recognize a variety of auditory inputs, and tactile input and/or output devices (e.g., a vibrator arranged on a handheld probe for tactile feedback to the user). The user interface 350 may include any suitable number of displays 352, such as one or more passive displays (e.g., for displaying ultrasound images) and/or one or more touch screens, which may form part of the control panel 354. The display 352 may implement the display 214 of the evaluation workstation 210.

System 300 further includes local memory 330, which may be implemented by one or more memory devices arranged in any suitable combination. The memory 330 is configured to stores information 333 used or generated by the system 300. For example, the memory 330 may store executable instructions that configure the processor 340 to execute one or more of the functions associated therewith. The memory 330 may also store settings (e.g., acoustic imaging settings, tissue-specific presets (TSPs)), make and model of the scanner, physical parameters and/or other information about the scanner and any transducers connected to the scanner, acquired imaging data and any imaging-related information, such as measurements and reports, obtained and/or generated during an ultrasound exam, and log files 331, each recording the workflow of an exam performed with the ultrasound scanner. Various other types of information used or generated by the ultrasound scanner in use may be stored in the memory 330, some of which may be stored locally only temporarily, such as during and/or only until transfer to external storage. The memory 330 may also store additional information associated with operation of the ultrasound user performance evaluation tool, such as in embodiments in which the scanner is configured to implement the graphical ultrasound user performance evaluation tool described herein. In some embodiments, the memory 330 may implement the memory 216 of the evaluation workstation 210.

The ultrasound transducer probe (or simply ultrasound probe or transducer) 310 comprises a transducer array 314, optionally a beamformer (e.g., microbeamformer 316), one or more analog and digital components (e.g., for converting analog signals to digital signals and vice versa), and a communication interface (not shown) for communicating, via the communication link 311, signals between the transducer 310 and the base 320. The transducer array 314 is configured to transmit ultrasound signals (e.g., beams, waves) into a target region (e.g., into the patient's body) and receive echoes (e.g., received ultrasound signals) responsive to the transmitted ultrasound signals from the target region. The transducer 310 may include any suitable array of transducer elements which can be selectively activated to transmit and receive the ultrasound signals for generating images of the anatomy. A variety of transducer arrays may be used, e.g., linear arrays, curved arrays, or phased arrays. The transducer array 314, for example, can include a two-dimensional array (as shown) of transducer elements capable of scanning in both elevation and azimuth dimensions for 2D and/or 3D imaging. In some examples, the transducer array 314 may be coupled to a microbeamformer 316, which may be located in the ultrasound probe 310, and which may control the transmission and reception of signals by the transducer elements in the array 314. In some examples, the microbeamformer 316 may be coupled, e.g., by a probe cable or wirelessly, to a transmit/receive (T/R) switch 318, which switches between transmission and reception and protects the main beamformer 322 from high energy transmit signals. In some examples, for example in portable ultrasound systems, the T/R switch 318 and other electronic components of the system 300 that are shown in FIG. 1 as located in the base 320, may instead be included in the ultrasound probe 310. The transmission of ultrasonic signals from the transducer array 314, e.g., optionally under the control of the microbeamformer 316, may be directed by a transmit controller 324, which may be coupled to the T/R switch 318 and the main beamformer 322. The transmit controller 324 may control characteristics of the ultrasound signals transmitted by the transducer array 314, for example, amplitude, phase, and/or polarity of the waveform. The transmission of signals (i.e. acoustic energy) from the transducer array 314, under the control of transmit controller 324, occurs in accordance with acoustic settings, also referred to as imaging or acquisition settings, which may be manually controlled by the user (e.g., set via the user interface 350) and/or at least partially automatically controlled by a processor of the system 300. The transmit controller 324 may also control the direction in which beams are steered. Beams may be steered straight ahead from (orthogonal to) the transducer array 314, or at different angles for a wider field of view. The transmit controller 324 may be operatively coupled to the user interface 350, via which the system 200 receives user input. For example, the user may select whether transmit controller 324 causes the transducer array 314 to operate in a harmonic imaging mode, fundamental imaging mode, Doppler imaging mode, or a combination of imaging modes (e.g., interleaving different imaging modes). In some examples, the partially beamformed signals produced by the microbeamformer 316 may be coupled to the main beamformer 322 where partially beamformed signals from individual patches of transducer elements may be combined into a fully beamformed signal. In some examples, microbeamformer 316 can be omitted, and the transducer array 314 may be under the control of the main beamformer 322, which can then perform all beamforming of signals. The beamformed signals are coupled to signal processing circuitry (e.g., to the processor(s) 240) configured to produce ultrasound images of the patient's anatomy from the beamformed signals as they are acquired by and while scanning the patient.

The signal processing circuitry (e.g., processor(s) 340) includes a signal processor, which may be configured to process the received beamformed signal in various ways, e.g., including any suitable combination of bandpass filtering, decimation, I and Q component separation, and harmonic signal separation, to generate image data. The processing of signals performed by signal processor 326 may be different based, at least in part, on the imaging mode (e.g., B-mode, M-mode, Pulsed-Wave/Spectral Doppler, Power/Color Doppler, elastography, contrast-enhanced ultrasound (CEUS) imaging, microflow imaging (MFI) and others) to which the system 300 is set for imaging. For example, e.g., such as during B-mode imaging, the signal processor 326 may perform I/Q demodulation on the signal and then perform amplitude detection to extract amplitude data (e.g., A-lines) that can be arranged into a B-mode image. In the case of Doppler imaging, the signal processor 326 may perform additional or different combinations of filtering, spectrum analysis and/or flow estimation (e.g., Doppler or frequency shift estimation) to obtain suitable data for generating the selected type of images.

Following processing by signal processor 326, the image data is coupled to a scan converter 328 and/or a multiplanar reformatter 336. The scan converter 328 may be configured to arrange the data from the spatial relationship in which they were received to a desired image format so that the image data is presented on the display in the intended geometric format. For instance, data collected by a linear array transducer would be arranged into a rectangle or a trapezoid, whereas image data collected by a sector probe would be represented as a sector of a circle. As such, scan converter 328 is configured to arrange the image data from the spatial relationship in which they were received to the appropriate image format. The image data may be arranged by scan converter 328 into the appropriate two-dimensional (2D) format (e.g., 2D sector format), or three-dimensional (3D) format (e.g., a pyramidal or otherwise shaped format). The processor(s) may implement a multiplanar reformatter 336, which is configured to perform multiplanar reconstruction, e.g. by arranging data received from points in a common plane in a volumetric region into an image of that plane or slice, for example as described in U.S. Pat. No. 6,443,896 (Detmer). The scan converter 328 and multiplanar reformatter 336 may be implemented as one or more processors in some embodiments. A volume renderer 332 may generate an image (also referred to as a projection, render, or rendering) of the 3D dataset as viewed from a given reference point, e.g., as described in U.S. Pat. No. 6,530,885 (Entrekin et al.). The volume renderer 332 may be implemented by one or more processors. The volume renderer 332 may generate a render, such as a positive render or a negative render, by any known or future known technique such as surface rendering and maximum intensity rendering. The image data may be further enhanced, e.g., by image processor 334, through speckle reduction, signal compounding, spatial and temporal denoising, and contrast and intensity optimization. Numerous other signal and image processing techniques for generating images for various imaging modes have been developed and are well known and thus outside of the scope of the present invention. Thus for conciseness, these various techniques are not detailed herein and it will be understood that any suitable technique(s), currently know or later developed, for processing the acquired ultrasound signals to produce images for one or more desired imaging modes can be used without departing from the scope of the present disclosure.

As noted above, images acquired by the system 300 may be stored locally, in some cases temporarily, in the memory 330, which may be implemented by any suitable non-transitory computer readable medium (e.g., flash drive, disk drive). Other information stored in the memory 330 may include service log files 331 generated by the system 300. The information stored in memory 330 (e.g., service log files 331, image data, etc.) may be coupled, via one or more processors (e.g., system controller 338) to the user interface 350, e.g., for the presentation of images on the display, and/or to an external computing and/or storage system, such as via the external communication link 313, which may be any suitable wired or wireless communication link. The one or more processors 340 (e.g., system controller 338) may implement the functionality of the graphical ultrasound user performance evaluation tool described herein and may control the user interface 350 and communicate with memory 330 and/or external storage devices to implement one or more processes of the graphical ultrasound user performance evaluation tool.

In some examples of the ultrasound user performance evaluation system that is embodied on an ultrasound scanner, additional advantageous features may be provided. In some such embodiments, certain aspects of the evaluation process, such as the processing of the log file to identify certain events or performance metrics, may be performed in real-time while the exam is occurring. The evaluation system may be configured to display a training GUI (e.g., as a pop-up screen) during a live exam, which may provide real-time assistance to the ultrasound user. For example, the training screen may pop up for example when an abnormal feature in the log file is detected, including a very long idle time or a very long dead time as compared to the expected idle or dead time at that particular phase of an exam having the same upstream clinical context. In some embodiments, the training GUI may display a selected message appropriate for the situation, such as to instruct the user on how to resolve the problem. In some embodiments, the training GUI may be collaborative in that it may communicatively connect the scanner with a supervisor or expert user. Such a collaborative GUI may be implemented in the form of chat window or it may activate audio-visual components of the machine to enable live conversation between the collaborators (e.g., ultrasound user and expert/supervisor) during the exam. In other embodiments, the training GUI may additionally or alternatively function as a call button to summon a more experienced user for assistance. Various other advantageous features when implementing the ultrasound user evaluation tool directly on the scanner may be provided.

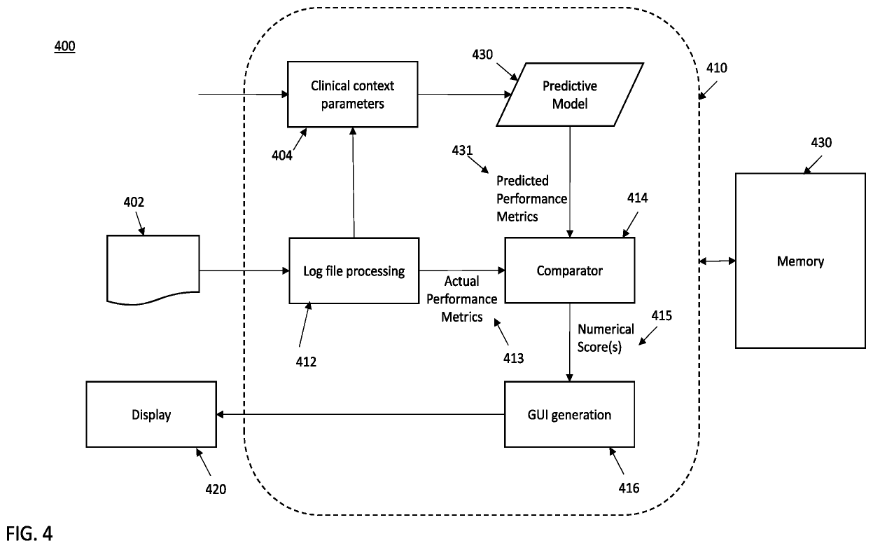

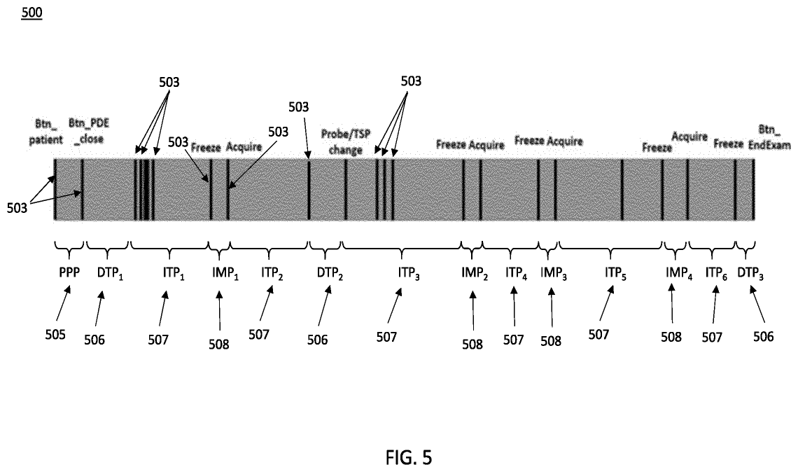

FIG. 4 shows components of a ultrasound user evaluation system 400 according to some embodiments of the present disclosure, which will be described with reference also to FIG. 5-8 illustrating graphical user interface screens of the ultrasound user performance tool and dashboard implemented by system 400. The ultrasound user evaluation system 400 of FIG. 4 may be used to implement the evaluation workstation 210 of FIG. 2. In some embodiments, the ultrasound user evaluation system 400 of FIG. 4 may, additionally or alternatively, be embodied in individual ones of the ultrasound imaging system 300 of FIG. 3, which may be part of a larger medical institution. In such examples the functionality of processor 410 may be implemented by one or more of the processors 340 of the imaging system 300, such that graphical displays of the ultrasound user evaluation system (e.g., the GUI screens of the dashboard in FIGS. 6-8) may be presented directly on the imaging system, such as at the completion of an exam. As shown in FIG. 4, the ultrasound user evaluation system 400 includes a processor 410 communicatively coupled to a display 420 and one or more memory devices 430. The memory 430 stores various information for use by processor 430 when executing the ultrasound user evaluation tool (or application). For example, the memory 430 may store instructions for generating and displaying the various graphical elements of the dashboard, instructions for processing the log file(s) 402, etc.

The processor 410 is configured to receive an ultrasound machine log file 402. As described with reference to FIG. 2, the ultrasound machine log file (or simply log file) 402 is generated by an ultrasound imaging system (or scanner) during an ultrasound exam performed by a ultrasound user. The log file 402 records or logs events (e.g., user control selections (or button clicks) and associated settings applied to the scanner, identifying information about the scanner, patient, ultrasound user, and various machine status information) as they occur during an ultrasound exam while the ultrasound user operates the scanner. The term button click in the present context refers to any manipulation of a user control by the user (e.g., ultrasound user) irrespective of whether the control is a soft control or a hard control, and the particular configuration of the user control (e.g., slider type button, an On/Off type button, knob, a selectable icon or any other GUI widget). The log file 402 captures and records all manipulations of the system, including but not limited to settings changes, image captures and measurement recording, by the user through the system's user interface, and the time of occurrence of each event As such, the log file 402 provides a recording of the full timeline 500 of an ultrasound exam (see e.g., example in FIG. 5) performed by any given ultrasound user 204. As such, log files typically contain information about the exam workflow that is not otherwise available in other recorded media, e.g., image files acquired by the scanner and subsequently transferred to PACS. In accordance with the principles of the present invention, the processor 410 is configured to determine ultrasound user performance metrics based on the information recorded in the ultrasound machine log file(s) 402. Referring back to the exemplary operational environment in FIG. 2, the log file 402 may be received by the processor 410 directly from an ultrasound scanner (e.g., scanner 220) or it may be retrieved from a storage device that is not co-located with the imaging device (e.g., from external storage device(s) 232).

The processor 410 is configured, e.g., by executable instructions stored in memory (e.g., memory 430), to process the received log file 402 (at block 412) to extract the ultrasound user's actual performance metrics 413, compare a ultrasound user's performance metrics 413 to the performance metrics 431 (at block 414) to determine at least one ultrasound user performance score, and to graphically represent the ultrasound user performance score(s) (at block 416) on the display 420. Corresponding performance metrics to the actual metrics 413 extracted from the log file 402 may be obtained by processor 410 from a predictive model 430, and one or more numerical scores 415 may be generated based on the comparison of the actual to the predicted metrics. The predictive model 430 may generate the predicted (or expected performance) for any given upstream clinical context 404, which may be received by processor 410 and/or partially extracted (e.g., by processor 410) from the log file 402 or based on information contained in the log file 402. The log file 402 contains information (e.g., scanner button clicks and associated settings, and other machine status information) recorded during an ultrasound exam based on the user's operation of the scanner. As such, the log file 402 provides a recording of the full timeline of an ultrasound exam performed by any given ultrasound user 204

At block 412, the processor extracts a ultrasound user's actual performance in the form of actual performance metrics from the received log file 402. Various performance metrics may be determined from the events recorded in the log file. For example, metrics such as total idle time, total dead time, total patient preparation time, total exam time, total number of clicks and/or total number of clicks of certain type of button, frequency of selection of certain buttons, number of probe and/or TSP changes, etc. may be determined from exam timeline recorded in the log file. Referring also to FIG. 5, it can be seen that an exam workflow or timeline 500 may include or be segmented into different phases, including a patient preparation phase (PPP) 505, one or more dead time phases (DTP₁, DTP₂, . . . DTP_{M}) 506, one or more idle time phases (ITP₁, ITP₂, . . . ITP_{N}) 507, and one or more imaging phases (IMP₁, IMP₂, . . . IMP_{K}) 508. The total patient preparation time metric can thus be determined by determining the total duration of the patient preparation phase (PPP) 505. The total idle and dead time metrics can be determined by summing the duration of the dead time and idle time phases 506 and 507, respectively. Similarly, the total imaging time metric can be determined by summing the durations of all imaging phases 508. The total number and/or types of phases present in a given exam timeline may vary depending on the clinical context and thus predictions of expected performance metrics preferably take into consideration the particular clinical context of the exam for which a ultrasound user is evaluated. The duration of each phase can be determined based on the time attribute of the relevant recorded events. Referring to the visual representation of the exam timeline in FIG. 5, each vertical line represents an event (or button click) 503 recorded in the log file and associated with a timestamp, which may include the time and/or date when the event was logged. When processing the log file (e.g., at block 412), each event and its associated time may be extracted and temporarily recorded in a suitable data structure (e.g., a table). Other attributes (e.g., a value, such as the setting, associated with certain events) may also be recorded in the table. Certain other information obtained from the log file 402, such as ultrasound user identification information, patient identification information, scanner type information, may be extracted at block 412 and used (e.g., as clinical context parameters) in further processes (e.g., expected performance predictions) of the system 400.

Using the information extracted from the log file 402, the processor 410 determines performance metrics associated with the particular ultrasound user that conducted the exam recorded in the log file 402. The total exam time metric (referred to, in equation below, as the ExamDuration), which may be computed by the processor 410 by subtracting the time associated with the Exam Start event (e.g., the time of Btn_patient event in the example in FIG. 5) from the time of the Exam End event (e.g., time of Btn_endExam in FIG. 5). The patient preparation time metric, which corresponds to the duration of the PPP time interval in the example in FIG. 5 may be determined by subtracting the time of the Exam Start event from the Patient-form Close event (e.g., shown as the Btn_patient and Btn_PDE_Close exemplary events in FIG. 5). It will be understood that the specific event labels in the log files of different machine models may differ from the specific example in FIG. 5 and the labels provided there are merely illustrative of the given types of events being identified.

Idle time and dead time are phases during which active imaging (e.g., image and/or measurement recordings) is not occurring and thus often represent timing to be minimized for maximizing the efficiency of the exam workflow. Actual imaging time may be identified as the time between the occurrence of an Acquire event and the time of the immediately preceding Freeze event. Thus, the processor 410 may identify one or more imaging phases by identifying pairs of a Freeze event immediately followed by an Acquire event. The duration of each imaging phase (e.g., phases IMP₁ through IMP₄ in the example in FIG. 5) may be computed by subtracting the time associated with the Freeze event of a given pair from the time associated with the Acquire event of the pair.

The dead time may be identified as any portion of the exam during which the ultrasound probe is not acoustically coupled to the subject (e.g., the patient). Various algorithms exist that determine the state of the transducer (i.e., whether the transducer is acoustically coupled to the patient or not), such as the smart coupling algorithm for the PHILIPS L 14-3 transducer. Such algorithms are typically based on thresholding the acoustic energy returned from a certain depth to determine whether the transducer is coupled to skin or not. The transducer's state (e.g., acoustically coupled or not) can be automatically tracked by the ultrasound system and recorded as an even, e.g., with a binary such as 1 for coupled and 0 for uncoupled, in the log file. Alternatively, an image-based approach may be used to determine and record the state of acoustic coupling of the transducer, such as by processing the live video stream of imaging data and recording an event and associated timestamp when the image data indicates no-contact with the skin and vice versa recording another event as associated timestamp when acoustic coupling with the skin is again detected based on the image data in the live video stream. Accordingly, one or more dead-time phases may be identified based on the recorded changes in the acoustic coupling state of the transducer. The duration of each dead-time phase may be determined and the total dead time in a given exam may be computed by summing the duration of all dead-time phases of an exam.

The idle time may be defined as any portion of the exam which excludes imaging time, dead-time and the patient preparation time. The idle time may include time spent by the ultrasound user on setting up the machine (e.g., TSP selection, image quality adjustments), time manipulating the probe to an appropriate view, etc. Thus one or more idle time phases may be determined between any of the other phases. In some examples, the idle time may be extracted by identifying durations of time following an Acquire event and before the next Freeze event assuming that time is not interrupted by a decoupling of the probe from the patient (e.g., as may occur when changing the probe). The duration of each idle time phase may be determined and the total idle time of an exam may be computed by summing all idle time durations. Alternatively, the idle time may be computed by subtracting from the total exam time the total time take up by the other exam phases (e.g., the patient preparation phase, the imaging and dead-time phases, if any). In some embodiments, the processor 410 may be configured to determine additional performance metrics that can add additional context to the evaluation process. For example, idle time or dead time center of mass may be computed which may be used to determine in which portion of the exam (e.g., near the start or near the end) is there loss of time due to dead time or idle time. In one example, idle time center of mass which describes the center-of-mass of all idle time phases, with the exam timeline being mapped to the interval (0,1) may be computed as follows:

$$Idle time center of mass = \frac{1}{ExamDuration} ( ( {\sum }_{i = 1}^{{N}_{idle}} ( {t}_{e , i} - {t}_{s , i} ) \frac{{t}_{e , i} + {t}_{s , i}}{2} ) - ExamStart ) ,$$

where N_{Idle} is the number of idle-time phases and t_{s,i}, t_{e,i} are the start and end times of each idle time phase i, respectively. A value for the idle time center of mass which is below 0.5 implies that the major part of the idle time is concentrated in the first half of the exam, and conversely a value greater than 0.5 implies that a greater part of the idle time is in the last half of the exam. A similar calculation may be performed for the dead time. The center of mass calculation for the idle time or the dead time may provide an additional metric for the determination of the relevant performance score(s) and/or for selecting customized feedback to the user. Additionally, the various types of events may be counted (e.g., total Acquire events, total Freeze events, total imaging acquisition setting or TSP change events, etc.) and/or grouped into various categories to generate additional metrics on which the ultrasound user's performance is evaluated. For example, the total number of events of certain type (e.g., setting changes) may be used to determine ultrasound user's anatomical landmark identification score (e.g., score 630-5). The anatomical landmark identification score 630-5 represents the skill and efficiency of the user in finding the relevant anatomical landmark during imaging. The more changes to imaging settings, as captured by higher number of corresponding events recorded in the log file, the more likely that a ultrasound user struggled to efficiently find (e.g., early in the exam) the relevant landmark. In some embodiments, an anatomical landmark identification metric may be based, on the frequency count of image quality-related buttons while there is no change in the imaging mode, and while the idle time center of mass is below 0.5 (meaning the first half of the exam). The anatomical landmark identification score 630-5 may then be calculated as the percentage ratio of the actual metric as compared to the estimated predicted metric for a ultrasound user of a given experience level. Additionally or alternatively, frequency of certain events, specific settings applied, and other granular performance details may be displayed in one or more detailed reports and/or used for recognizing inefficient workflow patterns and providing customized feedback to the ultrasound user.

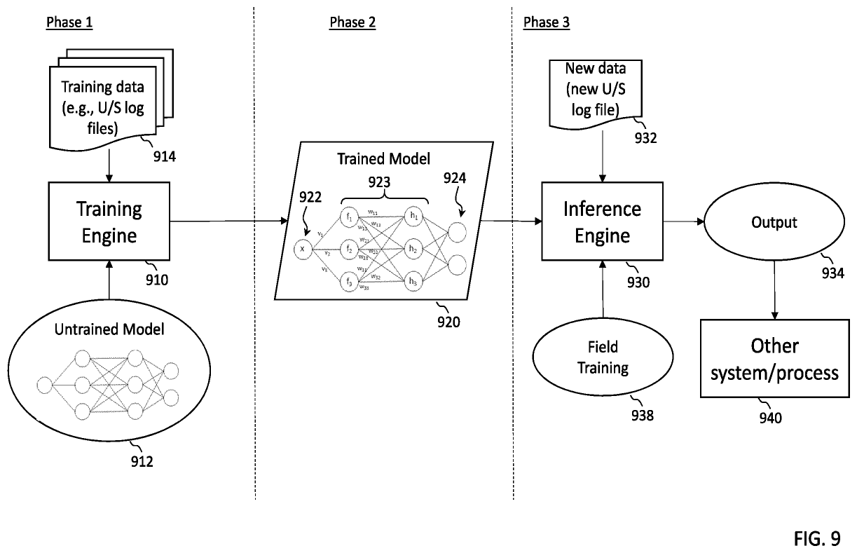

After the actual performance metrics 413 are extracted from the log file 402, the actual performance metrics 413 are compared, at block 414, to predict performance metrics 431 to obtain the ultrasound user's performance score(s). The predicted performance metrics 431 may be generated by a prediction model 430, which may be configured to output a respective set of predicted performance metrics for any one of a plurality of different ultrasound user of experience level (e.g., junior, mid-level, experience, expert, etc.), e.g., which may be specified by the user in some embodiments. As such the predicted metrics 431 represent expected performance by a ultrasound user at the desired (e.g., user-specified) experience level. In some embodiments, the predictive model 430 may be implemented by one or more analytical models (e.g., regression analysis model), by one or more neural networks of any suitable architecture (e.g., an artificial, convolutional, or recurrent neural network), or any combinations thereof. Neural networks of a suitable architecture may be used to output any of the numerical scores and/or qualitative (e.g., poor, good, excellent) scores of ultrasound user performance, the training of which will be described further below, e.g., with reference to FIG. 9.

The processor 410 may be configured to generate one or more performance scores in the form of numerical scores 415 based, at least in part, on the comparison of the ultrasound user's actual performance metrics 413 to the predicted (or expected) performance metrics 431. In some embodiments, the processor may additionally or alternatively generate one or more non-quantitative (e.g., a qualitative score such as low or poor, acceptable or good, and high or excellent) scores, such as image acquisition quality score 615 in the example in FIG. 6. The numerical scores 415 may be defined, in some embodiments, as the percentage ratios of respective ones of the actual performance metrics to the respective predicted performance metric of the experienced ultrasound user for the same clinical context. For example, an actual total dead time metric of 12 minutes when compared to a predicted total dead time metric of 10 minutes would yield a performance score of 83% for dead time management efficiency. In instances where the actual metric is as good as or outperforms the corresponding expected metric, a score of 100% may be generated. For any scores where the actual metrics are significantly below the expected metrics, such as at a below 50% performance, additional visual cues, such as color-coding, may be provided to the user via the graphical evaluation dashboard.

The performance scores (e.g., numerical scores 415 and/or qualitative scores and various visual cues) may be arranged on a graphical user interface (GUI) for display, as shown at block 416 in FIG. 5. The GUI generation (block 416) may include organizing scores by type and displaying the different types of efficiency scores in different display areas of the dashboard. The GUI generation (block 416) may further include applying visual cues, such as color, which may in some embodiments be associated with a non-numerical graphic, such as a dial graphic or any other suitable graphic that represents the associated numerical score. In some embodiments, the GUI generation further includes the preparation of customized feedback based on the determined performance score(s). A collection of different feedback messages may be stored in memory 430, among other information, and the processor 410 may select, at block 416, the appropriate subset of feedback messages based on the determined scores. The various GUI elements of the graphical dashboard are then provided on the display 420 for consumption and/or further customization (e.g., ultrasound user level selection, etc.) by the user.

The ultrasound user evaluation system according to the present disclosure is configured to graphically represent the ultrasound user performance scores in a graphical user interface (GUI) 600, also referred to as ultrasound user performance dashboard 600, an example of which is shown in FIG. 6. The information presented via the GUI or dashboard 600 may be provided on one or more GUI screens or windows, such as the GUI screen 610 in FIG. 6, and optionally in additional screens 710 and/or 810. In some embodiments, the ultrasound user performance dashboard 600 displays at ultrasound user performance score(s) 630, at least some of which may be quantitative and are derived from the information recorded in the log file. In the example in FIG. 6, the dashboard 600 is configured to display a first score 630-1 which indicates the ultrasound user's performance with respect to patient preparation efficiency, a second score 630-2 which indicates the ultrasound user's performance with respect to dead time management, and a third score 630-3 indicating the ultrasound user's idle time management performance. The dashboard 600 may further present a fourth score 630-4 which indicates the ultrasound user's overall exam efficiency. Numerical performance scores may be provided as a percent value, a value ranging between a predetermined minimum and maximum scores (e.g., a rating between 1 and 5, or other suitable numerical value. The dashboard 600 may be configured to graphically represent the performance score(s) in some cases displaying both the numerical score 632 and a non-numerical graphic 634 visually representing the numerical score, or it may be configured to display either of the numerical score 632 or graphic 634 without the other. In some embodiments, multiple performance scores 630 may be generated for different assessment categories of the ultrasound user's performance. For example, the system may determine exam efficiency scores (e.g., scores 630-1 through 630-4) which may substantially focus on the timing of completion of certain tasks and/or the minimization of lost time (e.g., through dead time or idle time). The dashboard 600 may present a fifth score 630-5, also referred to as anatomical information efficiency scores, which indicates the ultrasound user's skill/efficiency in identifying anatomical information (e.g., landmark identification, image and/or measurement acquisition, etc.). The system may also track total number of button clicks (e.g., setting changes, probe changes, freeze/capture/acquire events, etc.) and may present yet another score 630-6 which indicates the ultrasound user's efficiency as measured purely based on button clicks count. As previously mentioned, some or all of the scores presented on the dashboard 600 may be heavily dependent upon the type of exam being performed, the ultrasound scanner model, and other clinical context parameters, which are taken into account in the score determination process.

In some embodiments, the dashboard 600 is configured to group the scores 630 on the display in a manner that may be more intuitive and/or visually easy for the user to understand, which may improve the user experience. For example, the GUI screen 600 may be divided into multiple display areas. A first display area 612 may display one or more scores associated with exam efficiency (e.g., scores 630-1 through 630-4). A second display area 614 may display one or more scores associated with anatomical information efficiency (e.g., scores 630-5 and 630-6). Additional performance scores and/or display areas may be provided by the dashboard 600 in other embodiments. In some embodiments, the dashboard may provide an image acquisition quality score 615, which may be presented in yet another display area 616. The image acquisition quality score 615 and any other ultrasound user performance score may be presented non-quantitatively. For example, in the case of the image acquisition quality score 615, the score may be graphically represented by a descriptive word string and/or color to convey the ultrasound user's performance with respect to image acquisition quality. For example, as shown in FIG. 6, the image acquisition quality score 615 may be provided by the displaying, in the display area 616, a performance-descriptive word (e.g., poor, moderate, excellent), which may optionally be color encoded and/or in some embodiments, it may be presented as highlighting of the appropriate one of the plurality of available and displayed scores. In some embodiments, a performance score such as the acquisition quality score 615, may be represented, for example, as a bar with a low, medium, and high levels to indicate level of performance, and where each level is optionally coded in a different color. Alternatively, simply color may be used to represent the quality of performance (e.g., red for low or poor, orange for medium or satisfactory, and green for high or excellent).

In some embodiments, the dashboard 600 is configured to provide feedback 617 which is customized for the particular ultrasound user based on the one or more performance scores 630 presented on the dashboard. The customized feedback 617 may be presented in yet another display area 618, and the feedback itself may present positive feedback and/or negative/constructive feedback, which optionally may be color-coded (e.g., green for positive and red for constructive). Based on the performance scores 630, the processor (e.g., processor 410 or processor 212) may customize the feedback 617 for display in area 618, such as by selecting one or more feedback messages from a plurality of messages stored in memory. A collection of different messages (e.g., constructive feedback) may be stored in memory and associated (e.g., via a lookup table) with different scores with given score thresholds such that once the performance scores are determined, the processor can select for display the appropriate message(s) from the collection of stored messages that correspond to the particular determined score(s). Any of the display areas may be delineated from other display areas graphically or indirectly visually (e.g., by the grouping or clustering of associated information in a different portion of the screen 610.

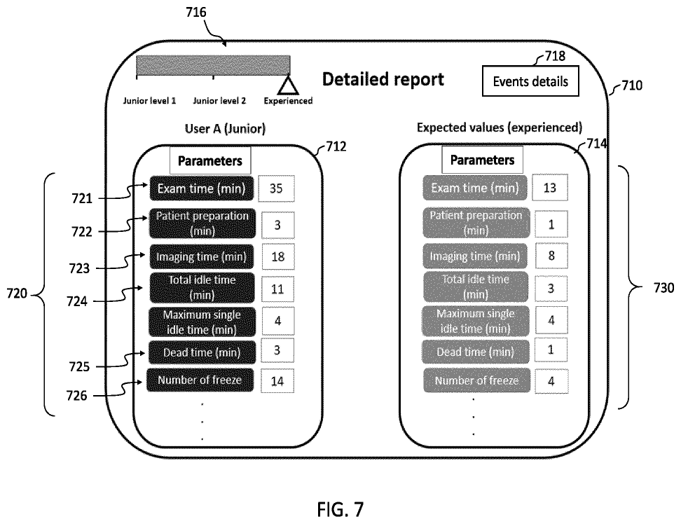

The dashboard 600 may include one or more user controls or widgets (e.g., drill-down widget 620, evaluation period widget 626, etc.) which may be selectable by a user (e.g., the ultrasound user or an evaluator other than the ultrasound user) to tailor the information displayed on the screen 610 and/or to invoke additional screens of the dashboard. For example, a first user control 620, which is also referred to herein as a first drill-down widget 620, may be provided in a ultrasound user performance summary screen (e.g., the GUI screen 610). Upon selection of the first user control 620, the dashboard 600 provides additional, more detailed information about the performance metrics on which the one or more scores 630 are based. This additional information may be presented in a separate GUI screen, such as GUI screen 710 shown in FIG. 7.

Referring now also to FIG. 7, the evaluation system may be configured to provide detailed information about the performance metrics based upon which the ultrasound user's performance scores were determined, such as upon selecting of an appropriate user control (e.g., widget 620). FIG. 7 shows one example of a GUI screen 710, also referred to as Detailed Report screen 710, that can be displayed responsive to clicking on widget (e.g. button) 620. In the GUI screen 710, the user can view details about the various events extracted from the ultrasound user's log file such as in the form of individual performance metrics 720, which may optionally be displayed concurrently (e.g., side by side) with the corresponding predicted values (e.g., as provided by model 430). For example, individual ones of the actual performance metrics 720 such as total exam time 721, patient preparation phase duration 722, total imaging time 723, total idle time 724, total dead time 725, total button clicks 726 of a particular type (e.g., number of freezes, acquires, gain changes, etc.) and others may be individually detailed in the GUI screen 710, such as in a Ultrasound user Metrics display area 712. The corresponding predicted performance metrics 730 against which the actual performance metrics 720 were compared to generate the performance scores 630 in screen 610 may also be displayed, e.g., in an Expected metrics display area 714. In this way, the user (e.g., ultrasound user or evaluator) can visually inspect and identify specific areas of weakness and thus areas for improvement. The evaluation dashboard may enable the user to specify the experience level against which the particular ultrasound user should be compared, such as via a user control 716. The user control 716 may be configured to enable the user to specify a desired ultrasound user experience level, and upon selection of the desired ultrasound user experience level, a new set of predicted performance metrics 730 may be obtained (e.g., by processor 410) and the values in the display area 714 updated. The ultrasound user's performance scores 630 in the main summary screen may also be updated based on the specified ultrasound user experience level. The user control 716 may be implemented in any suitable manner such as by a slider control, as shown in FIG. 7, that allows the user to adjust the experience level between the available minimum and maximum values of the experience level. In other examples, the user control 716 may be implemented by a text input field, a drop-down menu, a dial, etc.

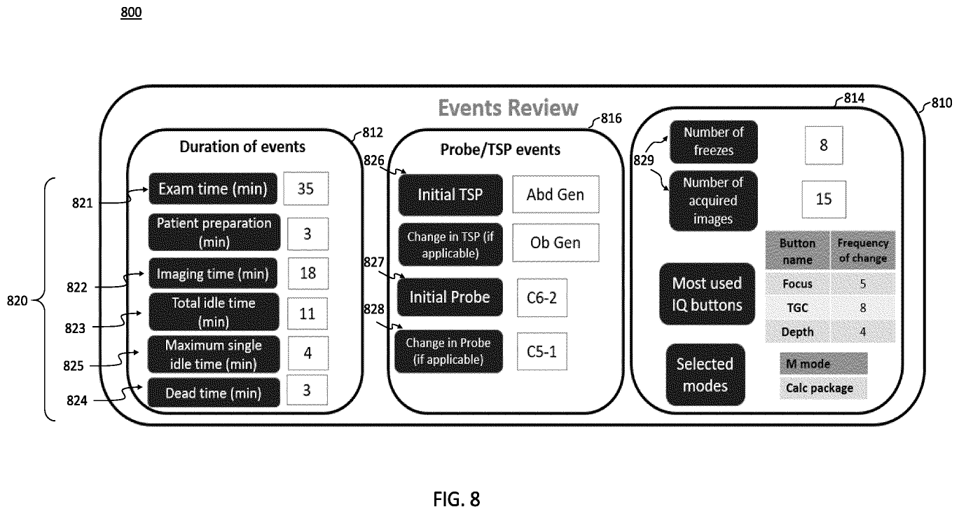

The ultrasound user performance evaluation tool may be configured to provide any desired level of details and information to enable adequate evaluation and/or opportunities for the training of ultrasound users. For example, to further facilitate training, additional details about the ultrasound user's performance may be made available, e.g., via another GUI screen 800 (FIG. 8), which may be invoked from the Detailed report screen 710 (e.g., via the user control 718 illustrated in FIG. 7 as a “Events details” button 718), or via a user control provided in a different GUI screen of the graphical performance evaluation tool (e.g., on a screen presented on the ultrasound machine at the completion of an exam). The GUI screen 800, which may also be referred to herein as Events Review report or self-assessment applet, may present detailed information about the number and types of different events recorded in the log file, as well as times/durations of the various phases of the exam. The latter information may not be directly available in the log file but is obtained through processing of the log file as described earlier. The format of the self-assessment applet may be predefined and stored in the system or it may be user-customizable (e.g., by an administrator of the system) such as to add or remove event fields. As can be seen from the example in FIG. 8, information 820 about various events, many of which may be utilized by the evaluation tool when conducting ultrasound user evaluations can be presented for the easy review of a user. For example event timing and duration information such as exam time duration 821, imaging time 822, idle time 823, dead time 824, maximum single idle time 825 and/or maximum single dead time, initial TSP 826, number of TSP changes and/or additional TSPs selected, initial probe section 827 and/or any probe changes 828, and various information about button selections 829. For example, the total number of one or more categories of buttons (e.g., freeze, image capture, measurement recording, etc.) may be counted and presented to the user and further analysis on the use of buttons may be performed and presented such as frequency of use of buttons, etc. As with other GUI screens associated with the ultrasound user performance evaluation tool, different categories of events may be grouped into different display areas. For example, events generally associated with exam efficiency may be grouped in a first display area 812, while button counts and details about selected settings may be grouped into one or more additional display areas, namely second display area 814 and third display area 816, respectively.

Returning back to the main/summary screen of dashboard 600 in FIG. 6, the dashboard may further include a second user control 626, also referred to as evaluation period widget 626, which may enable the user to change the evaluation period for which performance scores are determined and displayed. For example, the user control 626 may configure to enable to user to specify the evaluation period such as between a single exam (e.g., the current exam for example when evaluation occurs contemporaneously with the completion of an exam, or to select an exam completed on a specified date) or multiple exams, such as occurring over a specified period of time (e.g., a month, 3 months, etc.). In the case of the latter, scores from the different exams may be averaged and the averages presented as the scores 630 on the dashboard. Additionally or alternatively, the performance scores may be displayed as trends (e.g., graphs showing changes of a given performance score over time). In some examples, the dashboard may include a user control 624, which may be active for certain users (e.g., an evaluator) to enables certain users to select from among a plurality of different ultrasound users of their organization. The user control 624 may be implemented in any suitable manner, such as via a text input field, a drop-down menu, etc. which receives as input the ultrasound user's name and/or unique identifier. Upon selection of a given ultrasound user, the information displayed in the dashboard may be automatically updated to show the scores of the last exam performed by that ultrasound user, or may default to some other selection with respect to evaluation period.

Any one or more of the display areas and any one or more of the performance scores, in any suitable combination, may be provided by a dashboard 600 according to various embodiments of the disclosure. For example, in some embodiments, the Anatomical Information display area 614, the Image Quality display area 616, the Feedback display area 618 or any other of the display areas may be omitted altogether. Additionally or alternatively, one or more of the scores 630-1 through 630-4 or 630-5 through 630-6 may be omitted from their respective display area or grouped differently with different scores or additional scores not included in this example. Also, the locations of the different display areas may be varied as may be visually pleasing or appropriate (such as when additional information is presented via screen 610).

As previously noted, one or more functions of the evaluation system processor (e.g., processor 212 or 410) such as the predictive model, may be implemented by a trained neural network. FIG. 9 shows a block diagram of a process and elements associated with training and deployment of a neural network (also referred to as trained model 920, as differentiated from an analytical model) in accordance with the principles of the present disclosure. The process shown in FIG. 9 may be used to train any of the neural networks described herein, such as a neural network implementing the functions of predictive model 430 in FIG. 4. The left-hand side of FIG. 9, phase 1, illustrates a training phase of a predictive model. To train the predictive model, training data 914, which may include numerous sets of annotated log files, associated clinical context parameters, or combinations thereof, may be provided as inputs in numerous rounds of training to an untrained (or only partially trained) neural network or model 912 of suitable architecture. The training may be performed by a training engine 910 configured to couple the training data, over time, to the selected untrained model to progressively refine the predictive performance of the trained model. The training process may involve the selection of a suitable architecture for the model 912, which may be a blank architecture (e.g., an architecture with defined layers and arrangement of nodes but without any previously trained weights) or a partially trained model, such as the inception networks, which may then be further tailored for classification of ultrasound images. The neural network may include an input layer 922, an output layer 924, and a plurality of hidden layers 923 operating between the input and output layers. The size, width, depth, capacity, and/or architecture of the network may vary. For example, the number of nodes of the input and output layers, as well as the number and node arrangement/connections of the hidden layers may differ in different embodiments, e.g., based upon the desired outputs and inputs which the neural network is trained to operate on. The neural network 920 may be hardware—(e.g., neurons are represented by physical components) or software-based (e.g., neurons and pathways implemented in a software application), and can use a variety of topologies and learning algorithms for training the neural network to produce the desired output. For example, a software-based neural network may be implemented using a processor (e.g., single- or multi-core CPU, a single GPU or GPU cluster, or multiple processors arranged for parallel-processing) configured to execute instructions, which may be stored in a computer-readable medium, and which when executed cause the processor to perform a machine-trained algorithm for receiving clinical context input(s) and generating an expected activity level of the non-scanning hand of a ultrasound user performing the ultrasound exam included within the input clinical context. The neural network 920 may be implemented, at least in part, in a computer-readable medium comprising executable instructions, which when executed by a processor, may cause the processor to perform a machine-trained algorithm to output expected activity levels for the non-scanning hand of a ultrasound user performing a particular exam.

The training phase may include the preparation of training data 914, such as extracting clinical context parameters and/or annotating log files from exams performed by ultrasound users at various experience levels. Numerous previously acquired log files may be pre-processed in a similar manner as described above with reference to processing steps of block 412 to extract various performance metrics from each file. This information may be used to annotate log files, e.g., in instances when training the network for a log file input. In other cases, when a network or portion thereof is trained to predict performance metrics for a given clinical context, the performance metrics extracted from the numerous existing log files may themselves constitute part of the training data. If training a network to classify images with respect to quality, the training data may include ultrasound images annotated as to quality, e.g., by an expert ultrasound user of the medical institution. Preferably the ground truth information for training a model to be deployed in a particular medical institution is obtained by annotations consistent with the standards and practices of that institution as there may be significant variations among institutions in the standard practices and expected performance of each such institution. Also, various networks or branches of a single network may be trained to output the metrics and/or overall performance scores for different experience levels such that, depending on the input at deployment, the appropriate set of outputs are generated by activating the appropriate network or branch thereof.

The untrained model 912 (e.g., blank weights) and training data 914 are provided to a training engine 910 (e.g., ADAM optimizer or any suitable training engine based upon the selected architecture) for training the model. Upon sufficient number of iterations (e.g., when the model performs consistently within an acceptable error), the model 920 is said to be trained (and thus also referred to as trained model 920) and ready for deployment, which is illustrated in the middle of FIG. 9, phase 2. As shown in FIG. 9, and based upon the selected architecture, the trained model includes an input layer 922, and output layer 924, and one or more hidden layers 923 which are configured to apply the set of weights refined through the training processes along propagation paths of the hidden layers.

As shown in the right-hand side of FIG. 9, or phase 3, the trained model 920 is applied (via inference engine 930) for analysis of new data 932, which is data that has not been presented to the model during the initial training (in phase 1). For example, the new data 932 may include new log files and/or clinical context parameters from subsequent ultrasound exams. The trained model 920 implemented via engine 930, which may be executed on a host system (e.g., evaluation workstation 210, the ultrasound imaging system 300, or on a remote computing system communicatively coupled to the evaluation workstation 210 and/or the ultrasound imaging system 300) is used to process the new data 932 in accordance with the training of the model 920 to provide an output 934 (e.g., one or more predicted performance metrics and/or one or more ultrasound user performance scores). The output 934 of the trained model generated in the field (e.g., applied at the point of care) may then be used by the system for further processes 940 performed by the system, such as generating and presenting the graphical performance dashboard, for example as shown in FIG. 6. The trained model implemented by inference engine 930 may be further trained in the field, as indicated by field training block 938 to further improve the performance of the trained model 920.

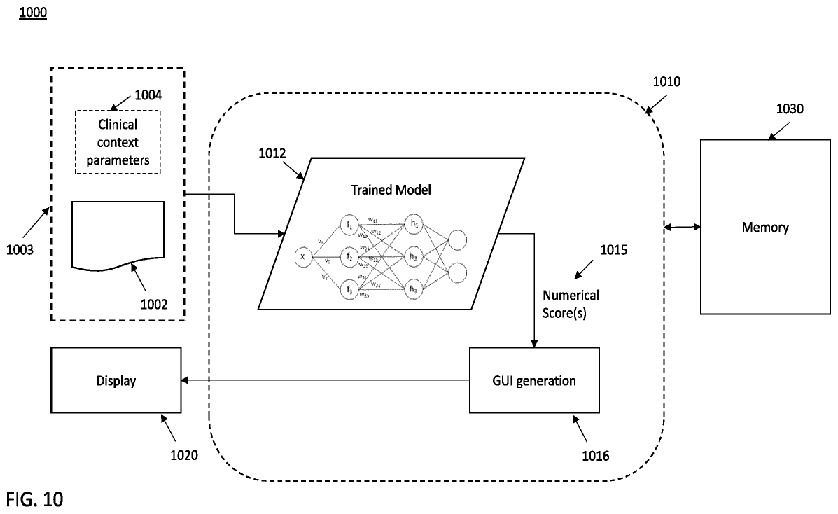

FIG. 10 shows a ultrasound user evaluation system 1000 according to further embodiments of the present disclosure. The system 1000 includes a processor 1010 in communication with a display 1020 and memory 1030. The memory 1030 stores information used by or generated by the processor 1010 for executing the graphical ultrasound user evaluation dashboard. The processor 1010 implements predictive model 1012, which may be provided by a trained neural network (e.g., implemented by a deep learning algorithm), is configured to receive as input an unknown ultrasound log file 1002 and output the performance score(s) 1015 of the ultrasound user. Optionally, the predictive model 1012 may also receive clinical context parameters 1004, which are not recorded in the log file (e.g., certain patient information such as clinical history, reason for exam, etc.). These clinical context parameters may be obtained (e.g., by processor 1010) from external sources (e.g., from RIS, PACS, EHR) and provided to the trained model 1012. The predictive model (e.g., trained neural network) 1012 is configured to estimate or predict the current ultrasound user's performance score(s) 1015 directly from the inputs 1003, e.g., the log file 1002 and/or clinical context 1004. This can be achieved by training the neural network with a suitable set of training data consisting of annotated log files of ultrasound users at different experience levels. In some embodiments, the predictive model 1012 may be trained to classify an incoming log file 1002 as representative of a particular experience level (e.g., a novice) and based upon this classification, the model 1012 may output a performance score 1015 as compared to a desired experience level (e.g., an expert). In the example in FIG. 10, steps associated with the extracting of actual performance metrics from the log file and comparing them against predicted performance metrics may be omitted as the predictive model is instead trained to directly estimate or quantify the current ultrasound user's performance without developing the underlying granularity (e.g., metrics) of the quantification process. Of course, in such cases, less information may be available downstream to the ultrasound user and/or evaluator for enhanced training of the ultrasound user. The processor 1010 is further configured, as indicated by GUI generation block 1016 to graphically present the ultrasound user performance scores obtained from the predictive model 1012 on the display 1020. The GUI generation 1016 may involve similar processes as described with reference to FIG. 4 (e.g., generating graphical representations of the one or more ultrasound user scores, organizing scores in different display areas, providing or enabling selectable areas on the display such as to provide various widgets for tailoring the display and/or for invoking additional display screens, customizing the feedback display are with comments selected based on the scores, etc.

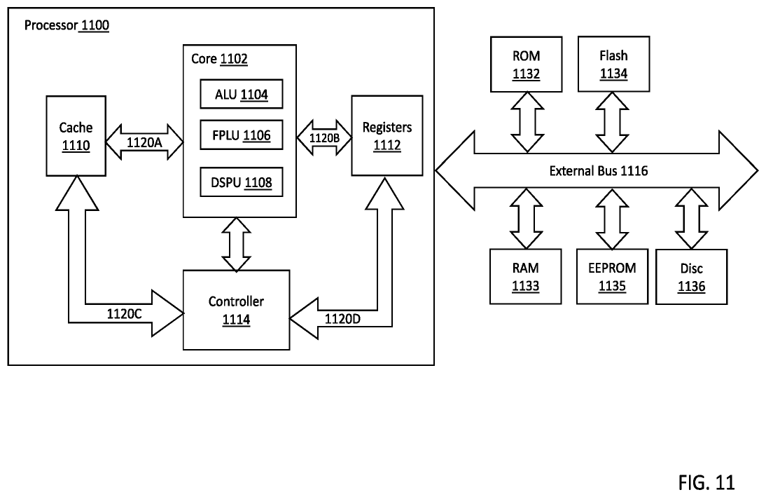

FIG. 11 is a block diagram illustrating an example processor 1100 according to the principles of the present disclosure. Processor 1100 may be used to implement one or more processors and/or controllers described herein, such as processor 212, any of the processors 340, or processors 410 or 1010. The processor 1100 may be any suitable processor type including, but not limited to, a microprocessor, a microcontroller, a digital signal processor (DSP), a field programmable array (FPGA) where the FPGA has been programmed to form a processor, a graphical processing unit (GPU), an application specific circuit (ASIC) where the ASIC has been designed to form a processor, or a combination thereof.

The processor 1100 may include one or more cores 1102. The core 1102 may include one or more arithmetic logic units (ALU) 1104. In some embodiments, the core 1102 may include a floating point logic unit (FPLU) 1106 and/or a digital signal processing unit (DSPU) 1108 in addition to or instead of the ALU 1104. The processor 1100 may include one or more registers 1112 communicatively coupled to the core 1102. The registers 1112 may be implemented using dedicated logic gate circuits (e.g., flip-flops) and/or any memory technology. In some embodiments, the registers 1112 may be implemented using static memory. The register may provide data, instructions and addresses to the core 1102. In some embodiments, processor 1100 may include one or more levels of cache memory 1110 communicatively coupled to the core 1102. The cache memory 1110 may provide computer-readable instructions to the core 1102 for execution. The cache memory 1110 may provide data for processing by the core 1102. In some embodiments, the computer-readable instructions may have been provided to the cache memory 1110 by a local memory, for example, local memory attached to the external bus 1116. The cache memory 1110 may be implemented with any suitable cache memory type, for example, metal-oxide semiconductor (MOS) memory such as static random access memory (SRAM), dynamic random access memory (DRAM), and/or any other suitable memory technology. The processor 1100 may include a controller 1114, which may control input to the processor 1100 from other processors and/or components included in a system (e.g., control panel 350, one or more I/O devices 211, or other processors of the system) and/or outputs from the processor 1100 to other processors and/or components included in the system (e.g., control panel 350, one or more I/O devices 211, or other processors of the system). Controller 1114 may control the data paths in the ALU 1104, FPLU 1106 and/or DSPU 1108. Controller 1114 may be implemented as one or more state machines, data paths and/or dedicated control logic. The gates of controller 1114 may be implemented as standalone gates, FPGA, ASIC or any other suitable technology.

The registers 1112 and the cache memory 1110 may communicate with controller 1114 and core 1102 via internal connections 1120A, 1120B, 1120C and 1120D. Internal connections may be implemented as a bus, multiplexor, crossbar switch, and/or any other suitable connection technology. Inputs and outputs for the processor 1100 may be provided via a bus 1116, which may include one or more conductive lines. The bus 1116 may be communicatively coupled to one or more components of processor 1100, for example, the controller 1114, cache memory 1110, and/or register 1112. The bus 1116 may be coupled to one or more components of the system, such as the display and control panel mentioned previously. The bus 1116 may be coupled to one or more external memories. The external memories may include Read Only Memory (ROM) 1132. ROM 1132 may be a masked ROM, Electronically Programmable Read Only Memory (EPROM) or any other suitable technology. The external memory may include Random Access Memory (RAM) 1133. RAM 1133 may be a static RAM, battery backed up static RAM, Dynamic RAM (DRAM) or any other suitable technology. The external memory may include Electrically Erasable Programmable Read Only Memory (EEPROM) 1135. The external memory may include Flash memory 1134. The external memory may include a magnetic storage device such as disc 1136. In some embodiments, the external memories may be included in a system, such as the local memory 216 of system 210 or external memory 232, or the local memory 330 of the imaging system shown in FIG. 3.

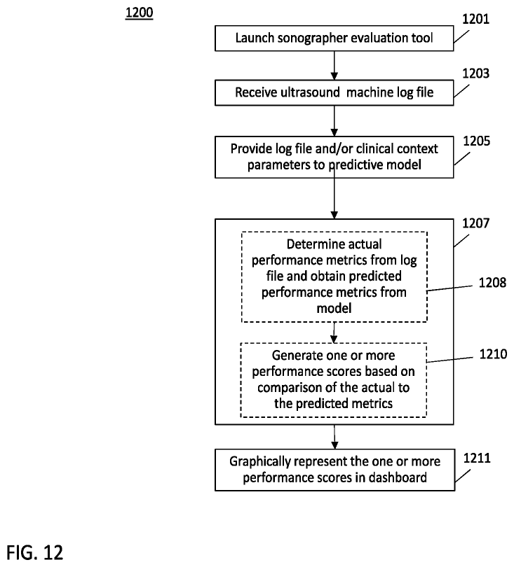

FIG. 12 shows a flow diagram of a computer-implemented method 1200 according to some embodiments of the present disclosure, which can be initiated, such as by launching ultrasound user evaluation app or tool, as shown in block 1201. The method 1200 includes receiving, by a processor in communication with a display, an ultrasound machine log file, as shown in block 1203. The log file and/or clinical context parameters are provided to a predictive model, as shown in block 1205 and using output from the predictive model, the processor determines one or more ultrasound user performance scores, based at least in part on the information recorded in the log file (see block 1207).

In some embodiments, as shown in block 1208, the process of determining the ultrasound user's performance scores includes determining actual performance metrics from the information in the log file and obtaining corresponding predicted metrics from a predictive model, as shown in block 1208. In some embodiments, to obtain the predicted metrics, clinical context parameters are provided to the predictive model, which may be implemented by a trained neural network as previously described. The predictive model generates predicted performance metrics for the specified clinical context and for a desired (e.g., user-specified) ultrasound user performance level. Then the ultrasound user's performance scores are determined based on a comparison between the actual and the predicted metrics, as shown in block 1210.

The method 1200 further includes graphically representing the one or more ultrasound user performance scores in one or more graphical user interface (GUI) screens of the ultrasound user evaluation tool (e.g., in a ultrasound user performance dashboard of the evaluation tool), as shown in block 1211. One or more use controls may be provided on the dashboard to enable a user to drill down and obtain additional information (e.g., detailed information about events, the actual (or recorded in log file) and the expected (or predicted by the model). In some embodiments, the method may include displaying, responsive to user request, the actual performance metrics concurrently with the predicted performance metrics. In some embodiments, the method may further include specifying, by user input, a desired ultrasound user experience level to be compared against and updating the predicted performance metrics on the display based on the user input.

In view of this disclosure, it is noted that the various methods and devices described herein can be implemented in hardware, software and firmware. Further, the various methods and parameters are included by way of example only and not in any limiting sense. In view of this disclosure, those of ordinary skill in the art can implement the present teachings in determining their own techniques and needed equipment to affect these techniques, while remaining within the scope of the invention. The functionality of one or more of the processors described herein may be incorporated into a fewer number or a single processing unit (e.g., a CPU) and may be implemented using application specific integrated circuits (ASICs) or general purpose processing circuits which are programmed responsive to executable instruction to perform the functions described herein.

A ultrasound user evaluation system (or an ultrasound imaging system that implements the ultrasound user evaluation system) according to the present disclosure may also include one or more programs which may be used with or associated with conventional imaging systems so that they may provide features and advantages of the present system. Certain additional advantages and features of this disclosure may be apparent to those skilled in the art upon studying the disclosure, or may be experienced by persons employing the novel system and method of the present disclosure. While described in the context of ultrasound imaging, it will be appreciated that the invention can be implemented and configured for the evaluation of radiologist operating systems of other medical imaging modalities (e.g., magnetic resonance imaging (MRI), X-ray, computerized tomography (CT), etc.). All such medical imaging systems employ the use of system or service log files with record the interactions of the operator with the machine and thus the performance of the operator in these exams may similarly be evaluated and similarly compared to the expected performance of a more experienced radiologist in the same imaging modality. Thus the examples herein can be equally applicable and advantageous for standardizing performance evaluations in virtually any other medical imaging context.

Another advantage of the present systems and methods may be that conventional medical imaging systems can be easily upgraded to incorporate the features and advantages of the present systems, devices, and methods. Of course, it is to be appreciated that any one of the examples, embodiments or processes described herein may be combined with one or more other examples, embodiments and/or processes or be separated and/or performed amongst separate devices or device portions in accordance with the present systems, devices and methods. Finally, the above-discussion is intended to be merely illustrative of the present system and should not be construed as limiting the appended claims to any particular embodiment or group of embodiments. Thus, while the present system has been described in particular detail with reference to exemplary embodiments, it should also be appreciated that numerous modifications and alternative embodiments may be devised by those having ordinary skill in the art without departing from the broader and intended spirit and scope of the present system as set forth in the claims that follow. Accordingly, the specification and drawings are to be regarded in an illustrative manner and are not intended to limit the scope of the appended claims.

1. A ultrasound user performance evaluation system comprising:
a display; and
one or more processors in communication with the display and at least one memory which comprises computer-readable instructions which when executed cause the processor to:
generate one or more ultrasound user performance scores associated with a ultrasound user, the one or more ultrasound user performance scores based, at least in part, on information recorded in an ultrasound machine log file resulting from an ultrasound exam performed by the ultrasound user with an ultrasound scanner; and
display a ultrasound user performance dashboard configured to graphically represent the one or more ultrasound user performance scores.

2. The system of claim 1, wherein each of the one or more ultrasound user performance scores comprises a numerical score and wherein the ultrasound user performance dashboard is configured to display a graphic representing the numerical score in addition to or instead of displaying the numerical score.

3. The system of claim 2, wherein the ultrasound user performance dashboard comprises a graphical user interface (GUI) screen divided into a plurality of display areas selected from a first display area configured to display any ultrasound user performance scores associated with exam efficiency, a second display area configured to display any ultrasound user performance scores associated with anatomical information efficiency, and a third display area configured to display any ultrasound user performance scores associated with image quality.

4. The system of claim 3, wherein the GUI screen further comprises a third display area configured to display ultrasound user feedback customized based on the one or more ultrasound user performance scores.

5. The system of claim 1, wherein the processor is configured to provide the ultrasound machine log file as input to a trained neural network and obtain the one or more ultrasound user performance scores as output from the trained neural network.

6. The system of claim 1, wherein the processor is configured to:
determine actual ultrasound user performance metrics associated with the ultrasound user from the ultrasound machine log file;
obtain predicted ultrasound user performance metrics from a predictive model; and
compare the actual performance ultrasound user metrics with the predicted ultrasound user performance metrics to generate the one or more ultrasound user performance scores.

7. The system of claim 6, wherein the processor is configured to provide the ultrasound machine log file, one or more clinical context parameters associated with the ultrasound exam, or a combination thereof to the predictive model to obtain the predicted ultrasound user performance metrics.

8. The system of claim 7, wherein the one or more clinical context parameters are selected from patient age, patient body mass index (BM), patient type, nature or purpose of the ultrasound exam, and model of the ultrasound scanner.

9. The system of claim 6, wherein the predictive model is configured to generate a respective set of predicted ultrasound user performance metrics for each of a plurality of different ultrasound user experience levels responsive to user input specifying a desired ultrasound user experience level.

10. The system of claim 6, wherein the predictive model comprises a trained neural network.

11. The system of claim 6, wherein the actual ultrasound user performance metrics and the predicted ultrasound user performance metrics each comprise a plurality of actual and expected metrics, respectively, the metrics selected from total idle time, total dead time, total exam time, total patient preparation time, total number of button clicks, total number of button clicks of a given button type, and total number of acquisition settings changes.

12. The system of claim 6, wherein the ultrasound user performance dashboard comprises a user control configured, upon selection, to display one or more of the actual ultrasound user performance metrics concurrently with corresponding ones of the predicted ultrasound user performance metrics.

13. The system of claim 6, wherein the ultrasound user performance dashboard comprises a user control configured to enable a user to select a ultrasound user experience level against which the actual ultrasound user performance metrics are compared.

14. The system of claim 1, wherein the processor, the display and the memory are integrated into a workstation of a medical institution, the workstation being communicatively coupled, via a network, to a plurality of ultrasound scanners of the medical institution to receive respective ultrasound machine log files from any one of the plurality of ultrasound scanners.

15. The system of claim 1, wherein the processor, the display and the memory are part of the ultrasound scanner.

16. A method of providing performance evaluation of a ultrasound user, the method comprising:
receiving, by a processor in communication with a display, an ultrasound machine log file generated responsive to an exam performed by the ultrasound user with an ultrasound scanner;
providing at last one of the ultrasound machine log file or clinical context parameters of the exam to a predictive model;
using an output from the predictive model, determining one or more ultrasound user performance scores; and
graphically representing the one or more ultrasound user performance scores in a first graphical user interface (GUI) screen of a ultrasound user performance dashboard, the ultrasound user performance dashboard further comprising GUI widget for controlling information provided by the ultrasound user performance dashboard.

17. The method of claim 16 further comprising:
providing the clinical context parameters to a trained neural network to obtaining predicted performance metrics;
determining, by the processor, actual performance metrics of the ultrasound user from information recorded in the ultrasound machine log file; and
comparing the actual performance metrics to corresponding ones of the predicted performance metrics to generate the one or more ultrasound user performance scores.

18. The method of claim 17, wherein said determining the actual performance metrics comprises at least two of: determining a total idle time during the exam, determining a total dead time during the exam, determining a total duration of the exam, determining total imaging time of the exam, determining a total number of button clicks during the exam, and determining a total number of button clicks of a given type.

19. The method of claim 17 further comprising at least one of:
displaying, responsive to a user request, the actual performance metrics concurrently with the predicted performance metrics; and
specifying, by user input, a desired ultrasound user experience level to be compared against and updating the predicted performance metrics on the display based on the user input.

20. A non-transitory computer readable medium comprising computer-readable instructions, which when executed by one or more processors configured to access one or more ultrasound machine log files, cause the one or more processors to perform the method of claim 16.
